(19) United States
(12) Patent Application Publication
(10) Pub. No.: US 2006/0110745 A1
(43) Pub. Date: May 25, 2006

(54) Method for amplifying nucleic acids

(75) Inventors: Yoshihide Hayashizaki, Ibaraki (JP); Toshizo Hayashi, Tokyo (JP); Yasumasa Mitani, Hiroshima (JP)

Correspondence Address: FOLEY AND LARDNER LLP;SUITE 500, 3000 K STREET NW, WASHINGTON, DC, 20007, US

(73) Assignees: KABUSHIKI KAISHA DNAFORM; THE INSTITUTE OF PHYSICAL AND CHEMICAL RESEARCH

(21) Appl. No.: 11/123,223

(22) Filed: May 6, 2005

Related U.S. Application Data
(60) Provisional application No. 60/568,842, filed on May 7, 2004.

(51) Int. Cl. C12Q 1/68; C12P 19/34

(52) U.S. Cl. 435/6; 435/91.2

(57) ABSTRACT

The invention provides a sequence specific method for amplifying nucleic acids. More particularly, the invention provides a method for amplifying nucleic acid sequences which enables such sequences to be detected with high precision, rapidity and high specificity as compared to conventional methods. The present invention also provides a simple method for cloning nucleic acids, particularly, a rapid and simple method for amplifying alternative splicing forms synthesized by an alternative splicing which is performed in a process of preparing a matured mRNA from a DNA.

### FIELD OF THE INVENTION

The present invention relates to a sequence-specific method for amplifying nucleic acids. More particularly, the present invention provides a method for amplifying nucleic acid sequences which enables such sequences to be detected with high precision, rapidity and high specificity as compared with conventional methods. Further, the present invention provides a simple method for cloning nucleic acids, particularly, a rapid and simple method for amplifying alternative splicing forms synthesized by an alternative splicing which is performed in a process of preparing a matured mRNA from a DNA.

### BACKGROUND OF THE INVENTION

In recent years, techniques of detecting nucleic acids such as gene diagnosis, nucleic acid test for agricultural products and infectious disease diagnosis have been widely utilized. Various methods are known as a method for detecting nucleic acids for the purpose of such test and diagnosis. For example, there is a method of performing a polymerase chain reaction (PCR) using a primer containing a nucleic acid sequence to be tested, and investigating the presence or the absence of the amplified product, and a method of using a labeled probe which binds to a nucleic acid sequence to be tested. Further, there is a RT-PCR method and a ligase chain reaction method (LCR method) in addition to PCR which is most frequently utilized as a method for amplifying nucleic acid sequences to be tested. Further, as an isothermal amplification method which does not need complicated temperature adjustment as in PCR, a strand displacement amplification method (SDA method), a self retaining sequence amplification method (3SR method), a Qβ replicase method, a NASBA method, a LAMP method, an ICAN method, and a rolling circle method are known. Detecting techniques using these methods has been developed, and sold as test kits. However, these techniques have a problem in that 1) detection takes time, 2) the detection step is complicated, and 3) precision is low, and practical implementation is difficult in cases where rapidness and simplicity are required, such as infectious disease testing at airports, and testing of agricultural products in the field.

### SUMMARY OF THE INVENTION

An object of the present invention is to, upon amplification of a desired nucleic acid sequence, enhance rate, eliminate amplification of background or non-specific sequences, and enhance specificity of amplification of a desired sequence, and provide a means for detecting whether a desired nucleic acid sequence is contained in a specimen or not rapidly and at a better precision, based on the presence or the absence of an amplification product.

Accordingly, in one aspect of the present invention, there is provided a method for amplifying a double-stranded nucleic acid, which comprises incubating the double-stranded nucleic acid in a solution containing at least one kind of a primer complementary to a part of one or more loop parts of a stem loop structure, under a condition where the double-stranded nucleic acid has the stem loop structure.

In other aspect of the present invention, there is provided a method for amplifying a double-stranded nucleic acid, which comprises incubating the double-stranded nucleic acid in a solution containing at least one kind of a first primer and at least one kind of a second primer, under a condition where the double-stranded nucleic acid has a stem loop structure, wherein the first primer has a sequence complementary to a part of one or more loop parts of a stem loop structure and the second primer has a sequence complementary to an amplification product of the first primer.

In another aspect of the present invention, there is provided a method for amplifying a double-stranded nucleic acid, which comprises steps of:

ligating a nucleic acid having at least one stem loop structure with the double-stranded nucleic acid; and

incubating the double-stranded nucleic acid in a solution containing at least one kind of a primer complementary to the part of one or more loop parts of a stem loop structure, under a condition where the double-stranded nucleic acid has the stem loop structure.

In still another aspect of the present invention, there is provided a method for amplifying a double-stranded nucleic acid, which comprises steps of:

ligating a nucleic acid having one or more stem loop structures with the double-stranded nucleic acid; and

incubating the double-stranded nucleic acid in a solution containing at least one kind of a first primer and at least one kind of a second primer, under a condition where the double-stranded nucleic acid has the stem loop structure, wherein the first primer has a sequence complementary to a part of one or more loop parts of a stem loop structure and the second primer has a sequence complementary to an amplification product of the first primer.

In still another aspect of the present invention, there is provided a method for amplifying a nucleic acid, which comprises steps of:

ligating an oligonucleotide forming a stem loop structure to one or more terminuses of a double-stranded nucleic acid, wherein the oligonucleotide contains any or both of a sequence complementary to a part of a first strand constituting a double-stranded nucleic acid, and a sequence complementary to a part of a second strand, and wherein the double-stranded nucleic acid can complementarily bind to the oligonucleotide to a part of the first strand, a part of the second strand or both of them, respectively, to form a new stem loop structure specific for a target double-stranded nucleic acid; and

incubating the nucleic acid in a solution containing at least one kind of a primer complementary to a loop part of the new stem loop structure.

In still another aspect of the present invention, there is provided a method for amplifying a nucleic acid, which comprises steps of:

ligating an oligonucleotide forming a stem loop structure to one or more terminuses of a target double-stranded nucleic acid, wherein the oligonucleotide contains either a sequence complementary to a part of a first strand constituting the double-stranded nucleic acid or a sequence complementary to a part of a second strand, or both of them and wherein the double-stranded nucleic acid can complementarily bind to the oligonucleotide to a part of the first strand, a part of the second strand, or both of them, respectively, to form a new stem loop structure specific for the double-stranded nucleic acid; and

incubating the nucleic acid in a solution containing at least one kind of a first primer and at least one kind of a second primer, wherein the first primer has a sequence complementary to a loop part of the new stem loop structure, and the second primer has a sequence complementary to an amplification product of the first primer.

In still another aspect of the present invention, there is provided a method for amplifying a nucleic acid, which comprises steps of:

ligating an oligonucleotide forming a stem loop structure to at least one or more terminuses of a target double-stranded nucleic acid, wherein the oligonucleotide contains either a sequence complementary to a part of a first strand constituting the double-stranded nucleic acid, or a sequence complementary to a part of a second strand, or both of them and wherein the double-stranded nucleic acid can complementarily bind to the oligonucleotide to a part of the first strand, a part of the second strand, or both of them, respectively, to form a new stem loop structure specific for the target double-stranded nucleic acid; and

incubating the nucleic acid in a solution containing at least one kind of a primer which is complementary to either a part of a first strand or a part of a second strand of the double-stranded nucleic acid constituting the loop part of the new stem loop structure, or both of them.

In still another aspect of the present invention, there is provided a method for amplifying a nucleic acid, which comprises steps of:

ligating a second nucleic acid to at least one or more terminuses of a target double-stranded nucleic acid containing one or more places of a single-stranded part forming a loop in a part of the double-stranded nucleic acid to form a hairpin structure or a loop structure; and

incubating the target double-stranded nucleic acid with the second nucleic acid linked thereto in a solution containing at least one kind of a primer complementary to a single-stranded part forming a loop in the double-stranded nucleic acid, or a part forming a loop at a terminus.

In still another aspect of the present invention, there is provided a method for amplifying a nucleic acid, which comprises steps of:

ligating a second nucleic acid to at least one or more terminuses of a double-stranded nucleic acid containing one or more places of a single-stranded part forming a loop in a part of a target double-stranded nucleic acid to form a hairpin structure or a loop structure; and

incubating the target double-stranded nucleic acid with the second nucleic acid ligated thereto in a solution containing one or more kinds of a first primer and one or more kinds of a second primer, wherein the first primer is complementary to a single-stranded part forming a loop in the target double-stranded nucleic acid or a part forming a terminal loop, and the second primer has a sequence complementary to an amplification product of the first primer.

Instill another preferable embodiment, the double strand is derived from a double-stranded nucleic acid having a loop formed of complementary strands of two different nucleic acids which result from an alternative splicing. In another preferable embodiment, sequence information of two different nucleic acids which result from an alternative splicing from an amplified nucleic acid can be obtained.

In still another aspect of the present invention, there is provided an oligonucleotide comprising a sequence complementary to a part of a target nucleic acid, wherein the oligonucleotide can form a secondary structure having one or more stem loop structures with the target nucleic acid, after the oligonucleotide is ligated to a target nucleic acid.

In still another aspect of the present invention, there is provided an oligonucleotide comprising a sequence complementary to a part of a first strand constituting a target double-stranded nucleic acid, and a sequence complementary to a part of a second strand, wherein a secondary structure having a new stem loop structure can be formed by binding complementarily the oligonucleotide and a part of the first strand or a part of the second strand or both of them, respectively, after the corresponding end of the double-stranded nucleic acid and two end of the oligonucleotide are ligated.

These oligonucleotides can be preferably used in the method for amplifying a nucleic acid of the present invention.

Objects, features and advantages of the present invention will become apparent by the following detailed explanation. However, detailed explanation and Examples of the present invention are shown for illustration, and it should be understood that various variations and modifications obvious to a person skilled in the art by this detailed explanation are within the scope of the present invention.

### BRIEF DESCRIPTION OF THE DRAWINGS

FIG. 1 is a view showing one embodiment of the present invention.

FIG. 2 is a view showing a cleavage site of a restriction enzyme, of a dumbbell form-type product used in Example 1.

FIG. 3 is a photograph showing results of Example 1.

FIG. 4 is a photograph showing results of Example 2.

FIG. 5 is a conceptional view showing a method of Example 1.

FIG. 6 is a conceptional view showing a method of Example 2.

### DETAILED DESCRIPTION OF THE PREFERRED EMBODIMENTS

In the present invention, a sequence forming a stem loop (hereinafter, referred to as “linking oligonucleotide”) is ligated to a target sequence to form a template nucleic acid for amplification. That is, in the present invention, the linking oligonucleotide is ligated to a target sequence and a complementary sequence thereof to form an amplification template of a double-stranded nucleic acid.

In such a double-stranded nucleic acid, when the double-stranded structure is formed between the target sequence and the complementary sequence thereof, a single-stranded loop is formed at one terminus or two opposite terminuses of a double-stranded part. It is desirable that a loop is formed at the opposite terminuses of the double-stranded part. A structure having one loop at each of the opposite terminuses of the double-stranded part is referred to as dumbbell form.

Ligating of the linking oligonucleotide and the target double-stranded nucleic acid is chemically or enzymatically performed after mutual overhang terminal parts are hybridized. It is desirable that such ligating step is enzymatically performed by a ligase.

A primer can be designed so as to anneal to an arbitrary place of a ligated or linked double-stranded nucleic acid. For example, the primer can be designed so as to anneal to a part of the loop part or the stem part of a stem loop structure. From a viewpoint of efficiency of amplification, it is desirable to design the primer so that it anneals to a part of the loop part of the stem loop structure. The number of bases of a primer is not particularly limited as long as the primer anneals to a nucleic acid which is to be a template. As a primer to be annealed, one or more kinds may be used, and plural kinds of primer which anneal to plural sites of a linked double-stranded nucleic acid can be used. Amplification efficiency can be further enhanced by using a second primer having the same sequence as that of a part of a linked double-stranded nucleic acid in addition to a primer complementary to the linked double-stranded nucleic acid. A primer having the same sequence as that of a part of the double-stranded nucleic acid may have the same sequence as an arbitrary sequence of a linked double-stranded nucleic acid and, in terms of amplification efficiency, a primer having the same sequence as that of a part of the loop part of the stem loop structure is desirable.

A DNA polymerase used in a nucleic acid synthesizing method in accordance with the present invention may be any DNA polymerase as long as it has strand displacement activity (strand displacing ability), and any of normal temperature type, medium temperature type and heat resistant type can be preferably used. In addition, this DNA polymerase may be wild type or a variant to which a mutation is artificially added. Examples of such DNA polymerase include a Phi29 phage DNA polymerase. Other examples include a variant in which 5′→3′ exonuclease activity of a DNA polymerase derived from a thermophilic Bacillus bacterium such as Bacillus stearothermophilus (hereinafter, referred to as “B. st”) and Bacillus caldotenax (hereinafter, referred to as “B. ca”), and a Klenow fragment of a DNA polymerase I derived from E. coli has been deleted. Further examples include a Vent DNA polymerase, a Vent (Exo-) DNA polymerase, a DeepVent DNA polymerase, a DeepVent (Exo-) DNA polymerase, a MS-2 phage DNA polymerase, a Z-Taq DNA polymerase, a Pfu DNA polymerase, a Pfu turbo DNA polymerase, a KOD DNA polymerase, a 9°Nm DNA polymerase, and a Therminator DNA polymerase. In order to improve heat resistance, it is possible to add trehalose or the like, or in order to stabilize an enzyme, it is possible to add glycerol or the like. Further, when the desired nucleic acid is a RNA, it is preferable to use a Bca (exo-) DNA polymerase having strong reverse transcriptase activity. When reverse transcriptase activity is weak, it is desirable to conbine these enzymes and M-MuLV Reverse Transcriptase or the like having reverse transcriptase activity.

The present invention may be utilized when one wants to detect an arbitrary sequence in a genome. In the present invention, it is possible to remarkably enhance a priming efficiency of a primer and, consequently, increase an amplification rate and enhance specificity. Due to high amplification specificity in accordance with the present invention, SNP (single base polymorphism) can be detected. Further, by adding a second primer having a sequence complementary to this amplified nucleic acid, a target sequence may be amplified exponentially.

In addition, in the present invention, the linking oligonucleotide is ligated or otherwise linked to the opposite terminuses of a straight chain double-stranded nucleic acid, and may be utilized in amplification. In this case, by performing the amplification reaction using a primer having a sequence complementary to the stem loop part, it becomes possible to enhance the rate of synthesizing a single-stranded long chain nucleic acid in which respective chains of DNAs are alternately bound, and has become possible to simply amplify without thermal denaturation which was necessary in the method described in WO 01/040516.

Further, in the present invention, the linking oligonucleotide can be designed so that an amplification reaction is commenced only when the linking oligonucleotide is precisely linked to the target sequence. Thereby, only the target nucleic acid can be selectively amplified from a mixture of plural kinds of nucleic acid molecules and, by measuring the presence or the absence of this amplification reaction, it becomes possible to detect the target nucleic acid contained in a sample.

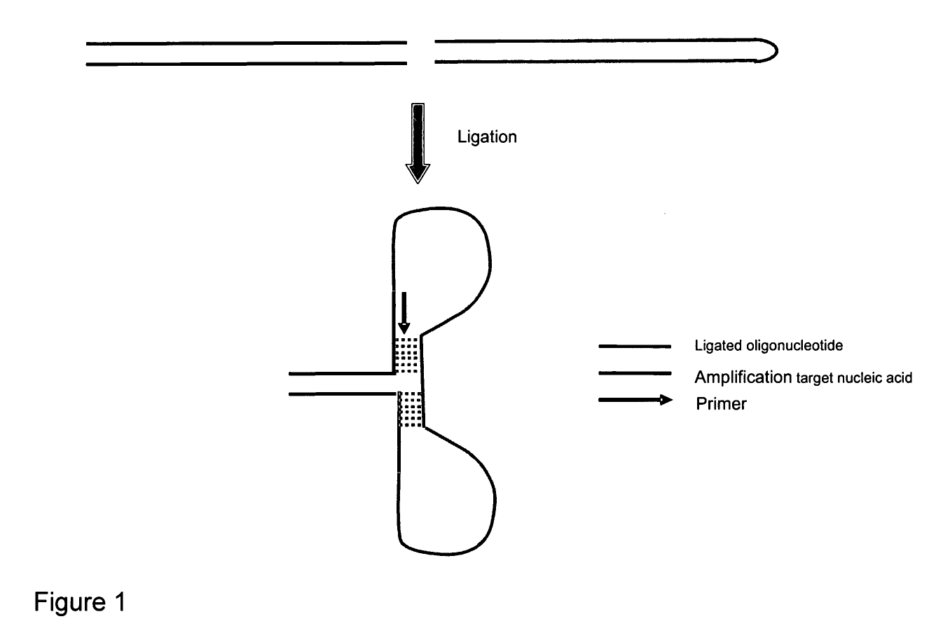

The specific design of this linking oligonucleotide having enhanced specificity is shown, for example, in FIG. 1. When the linking oligonucleotide is linked with a molecule other than the target nucleic acid, an erroneously linked molecule is amplified by rolling circle amplification, and specific amplification or detection of the target nucleic acid becomes difficult. In order to prevent such non-specific amplification, the linking oligonucleotide is designed so that the terminal sequence of the target nucleic acid makes up a part of the loop part of the stem loop after a target nucleic acid and the linking oligonucleotide are ligated. Unless a linking oligonucleotide and a target nucleic acid are ligated, a stem loop is not formed. Further utilizes is a primer for amplification having a sequence complementary to the terminal sequence of the target nucleic acid forming the loop part of the stem loop formed after the ligation or, preferably, a part of the loop part.

In addition, a plurality of primers for amplification may be used, but by utilizing a primer having a sequence complementary to the target sequence, specificity of amplification may be also enhanced. In addition, by incorporating a restriction enzyme recognition sequence into the linking oligonucleotide sequence in advance, a long chain nucleic acid molecule synthesized by the amplification reaction may be cut and degraded into nucleic acid molecules of the same length.

Further, by applying this method, an alternatively spliced form may be specifically amplified. Alternative splicing is a mechanism for synthesizing a plurality of different proteins from one locus, and it is known that a protein having different physiological activity or a protein which is the cause of a disease is synthesized in many cases. Therefore, alternative splicing is gathering a lot of attention. Several methods are known for collecting two kinds of spliced forms in the form of a double-stranded nucleic acid from a plurality of alternatively spliced forms produced from the same locus. In this double-stranded nucleic acid, an exon, which is a subject of alternative splicing, forms a loop, taking the form of a single strand. When a double-stranded nucleic acid obtained from two kinds of different alternatively spliced forms is amplified using the aforementioned method and using a primer having a sequence complementary to a sequence of the exon forming a loop, it becomes possible to specifically amplify an alternatively spliced form of a desired locus.

The present invention has been generally explained above and will be more specifically explained below by way of Examples. However, Examples are only for the purpose of explanation, and it is not intended to restrict the scope of the present invention to these Examples.

### EXAMPLES

### Example 1

### Linking of a Loop Cassette, and Amplification Using

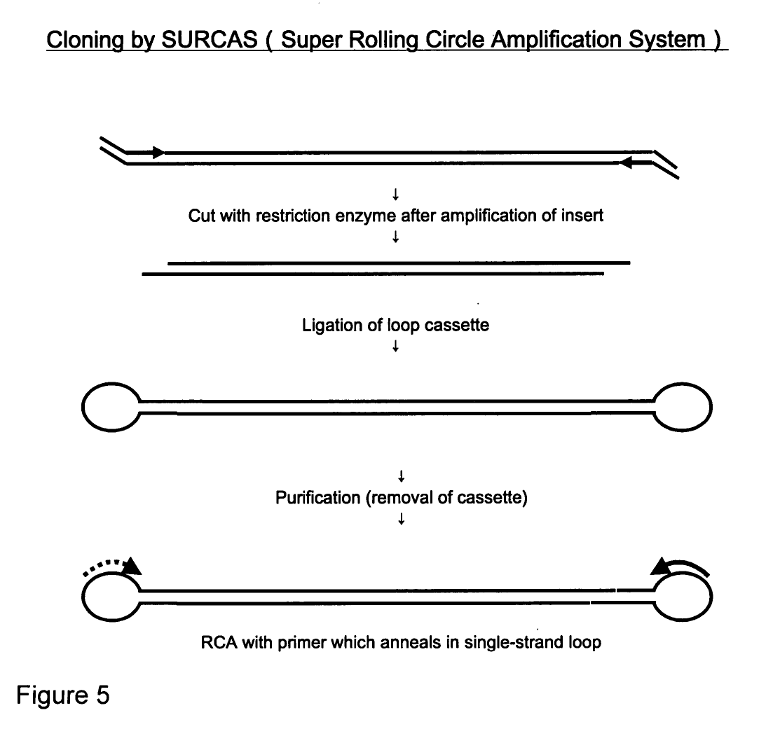

the same as a template By the SURCAS method (Super Rolling Circle Amplification System) shown in FIG. 5, a mouse musculus achaete-scute complex homolog-like 3 (Drosophila) (Ascl3) gene, ID Number: NM_{—}020051 was amplified using a mouse genome DNA as a template. The sequence of an insert is shown below (SEQ ID NO:1). An underlined part is a sequence which anneals to a 3′ terminal side of a primer, and a restriction enzyme (BamHI) cleaving site is shaded.

[image]

Amplification was performed using primers shown below.

(SEQ ID NO:2)

YH-F1:
5′ATGCGCGGACCCAGATTGCTGGATGGACACCAGAAGCTACCC

(SEQ ID NO:3)

YH-R1:
5′GCTGCGGCACCCAACAGAATGGTCAAATGACTCTCAGAGCCG

In the primer sequences, the underlined sequence is a sequence which anneals to the underlined sequence of the insert. A BstXI restriction enzyme recognition sequence (bold letter part) was added to the 5′ region of each primer. The synthesis of primers for amplification was carried out by Invitrogen Corporation.

The insert was amplified by PCR using these primers. The PCR reaction solution and the number of cycles are given as follows.

<Composition of PCR Reaction Solution>

| Component | Final | |
|---|---|---|
| 10Xbuffer | 1X | |
| MgCl₂ | 2.5 | mM |
| dNTPs | 200 | μM |
| Primer F | 0.2 | μM |
| Primer R | 0.2 | μM |
| Template | 500 | ng |
| AmpliTaq | 1.25 | U |
| H₂O | up to 25 | μl |

94° C.; 2 minutes, (95° C.; 30 seconds, 65° C.; 1 minute, 72° C.; 1 minute), 35 cycles

After PCR amplification, unreacted primers were removed by Promega Wizard® RSV Gel and PCR Clean-Up system to purify the desired amplification product.

The terminus of the purified amplification product (insert part) was subjected to restriction enzyme treatment with BstXI. The composition of a reaction reagent is as follows. Restriction enzyme treatment was performed at 50° C. for 90 minutes.

<Composition of Reaction Reagent>

| | | |
|---|---|---|
| BstXI Buffer | 5 | μl |
| BstXI | 1 | μl |
| Purified amplification product | 10 | μl |
| dH₂O | up to 50 | μl |

The amplification product whose terminus had been cut with a restriction enzyme was purified using Promega Wizard® SV Gel and PCR Clean-Up system.

The amplification product after purification was ligated to the loop cassette. The sequence of the loop cassette is shown below. This loop cassette is a 5′ terminal phosphorylated oligonucleotide. The underlined part is a loop part. In the loop cassette, the bold letter sequence in the loop indicated by the underlined part is a sequence which anneals to a loop primer. Each loop cassette was designed so that a 3′ terminus had an overhang by four bases (indicated by bold letter).

The amplification product and a loop cassette were ligated by treatment with a reaction reagent shown below at 16° C. for 90 minutes. Thereafter, Promega Wizard® SV Gel and PCR Clean-Up system was used to remove an unligated short chain loop cassette, and a dumbbell form-type product with a loop cassette linked thereto was purified.

<Loop Cassette Sequence>

(SEQ ID NO:4)

LOOP-F:
5′GCATCGACGGCATATGCCATAGCATTTTTATCCACGATCACCCGTCGA
TGCATTG3′

(SEQ ID NO:5)

LOOP-R:
5′GAGCCTAGCGCAGTACTGACGTTAAAGTATAGAGGTATCCCGCTAGGC
TCCAGA3′

Ligation Solution>

| | | |
|---|---|---|
| LOOP-F (10 uM) | 1 | μl |
| LOOP-R (10 uM) | 1 | μl |
| BstXI digested sample | 10 | μl |
| T4 DNA ligase buffer | 2 | μl |
| T4 DNALigase (NEB) | 2 | μl |
| dH₂O | up to 20 | μl |

Using the resulting dumbbell form-type product as a template, and using the following reagent composition, Rolling Circle Amplification was performed at a room temperature (25° C.) for 4 hours. A primer sequence is shown below. A loop primer set was designed so that it can anneal to a loop sequence, and a stem primer set was designed so that it can anneal to a stem sequence, respectively, and amplification was performed using each primer set.

<Primer Sequence for RCA>

Loop primer set

| | | |
|---|---|---|
| pBADF: | 5′ATGCCATAGCATTTTTATCC 3′ | (SEQ ID NO:6) |
| PGAL1: | 5′TACCTCTATACTTTAACGTC 3′ | (SEQ ID NO:7) |

Stem primer set

| | | |
|---|---|---|
| SF1: | 5′GATCACCCGTCGATGCATTG 3′ | (SEQ ID NO:8) |
| SR1: | 5′GTATCCCGCTAGGCTCCAGA 3′ | (SEQ ID NO:9) |

<Amplification Reagent Composition>

| | | |
|---|---|---|
| 10X buffer | 2.5 | μl |
| 100XBSA | 0.25 | μl |
| DMSO | 1.25 | μl |
| dNTPs (Final 140 μM) | 140 | μl |
| T4Gene32 (Amersham) | 0.5 | μl |
| Phi29Pol (NEB) | 2.0 | μl |
| Template (equivalent to about 10⁷ molecules) | 6.0 | μl |
| Each primer (Final 0.4 μM) | 2 | μl |
| H₂O | up to 25 | μl |

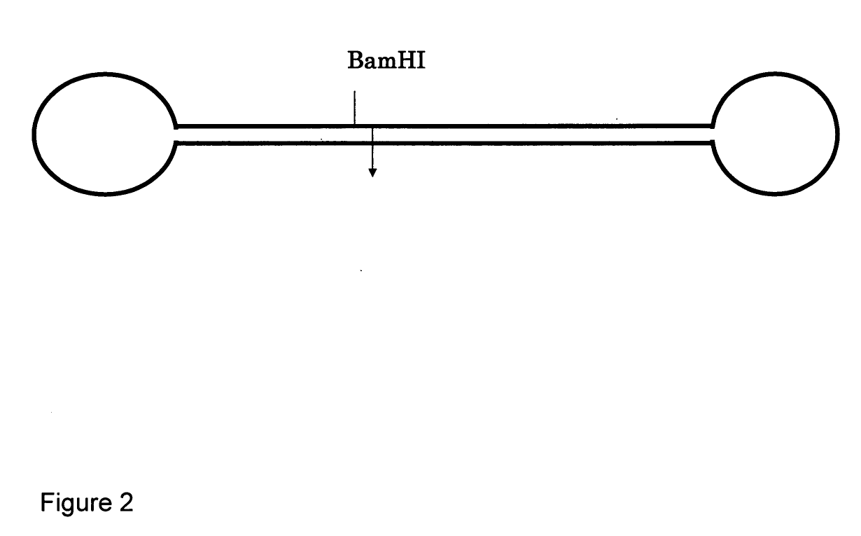

In order to confirm whether a desired sequence was amplified or not, restriction enzyme treatment was performed at 37° C. for 20 hours using a restriction enzyme BamHI. A cleavage site of a restriction enzyme of a dumbbell form-type product used in the present experiment is shown in FIG. 2.

| | | |
|---|---|---|
| Amplified product | 2 | μl |
| BamHI (TAKARA Co., Ltd. 10 unit) | 2.5 | μl |
| BufferK | 1 | μl |
| dH₂O | up to 10 | μl |

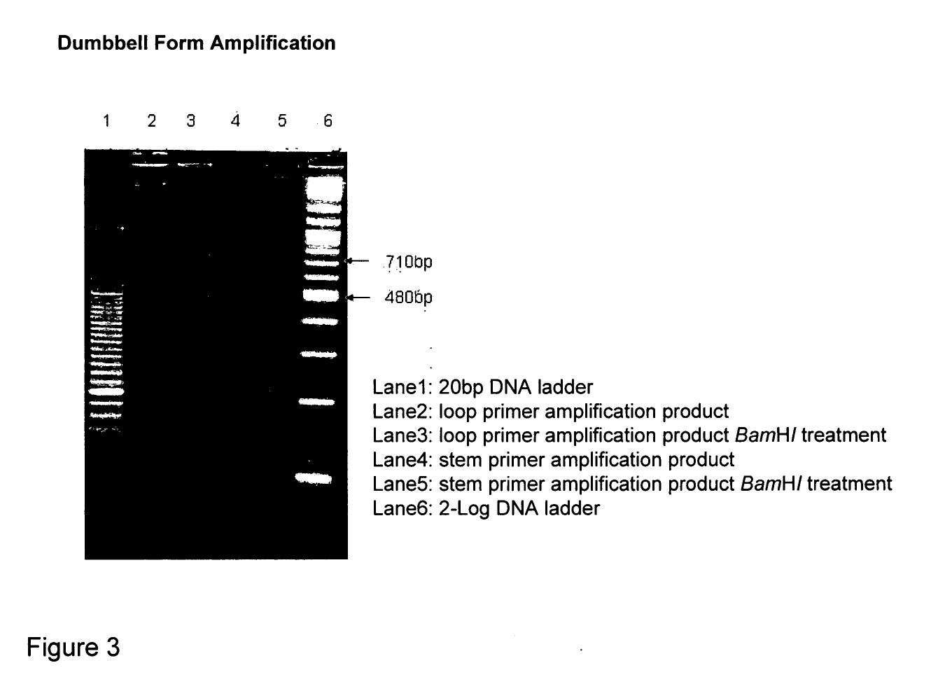

5 μl of the restriction enzyme-treated reaction solution was electrophoresed at 100 V for 80 minutes on a 1.5% nusieve 3:1 agarose gel (manufactured by TAKARA SHUZO Co., Ltd.). A gel after electrophoresis was stained with ethidium bromide (EtBr) to confirm a nucleic acid. Results are shown in FIG. 3. A sample of each lane is as follows.

Lane 1: 20 bp DNA Ladder size marker

Lane 2: amplification with Loop primer set, and then non-treatment with restriction enzyme

Lane 3: amplification with Loop primer set, and then treatment with BamHI

Lane 4: amplification with Stem primer set, and then non-treatment with restriction enzyme

Lane 5: amplification with Stem primer set, and then treatment with BamHI

Lane 6: 2-Log DNA Ladder size marker

After amplification using the loop primer set, lane 2 is non-treatment with a restriction enzyme, and the amplification product which had not been cleaved with a restriction enzyme was confirmed at about 10 Kbp. In lane 3, a nucleic acid was cleaved with BamHI, and a band was confirmed at about 480 bp and about 710 bp. These results were consistent with the size predicted from the restriction enzyme map shown in FIG. 2. From this, it was confirmed that a nucleic acid was amplified using an insert sequence linked with a loop cassette as a template. However, in the case of amplification with Stem primer set, the amplified product was not obtained and, even when restriction enzyme treatment was performed, a band of a desired size after cleavage was not obtained. In addition, when a dumbbell form-type linked double-stranded DNA was amplified, it was shown that it is not necessary to thermally denature to completely convert a template into a single strand, and the DNA is specifically amplified by using the loop primer set which provides a 3 terminus to a loop part forming a single strand.

### Example 2

### Clover Leaf Amplification

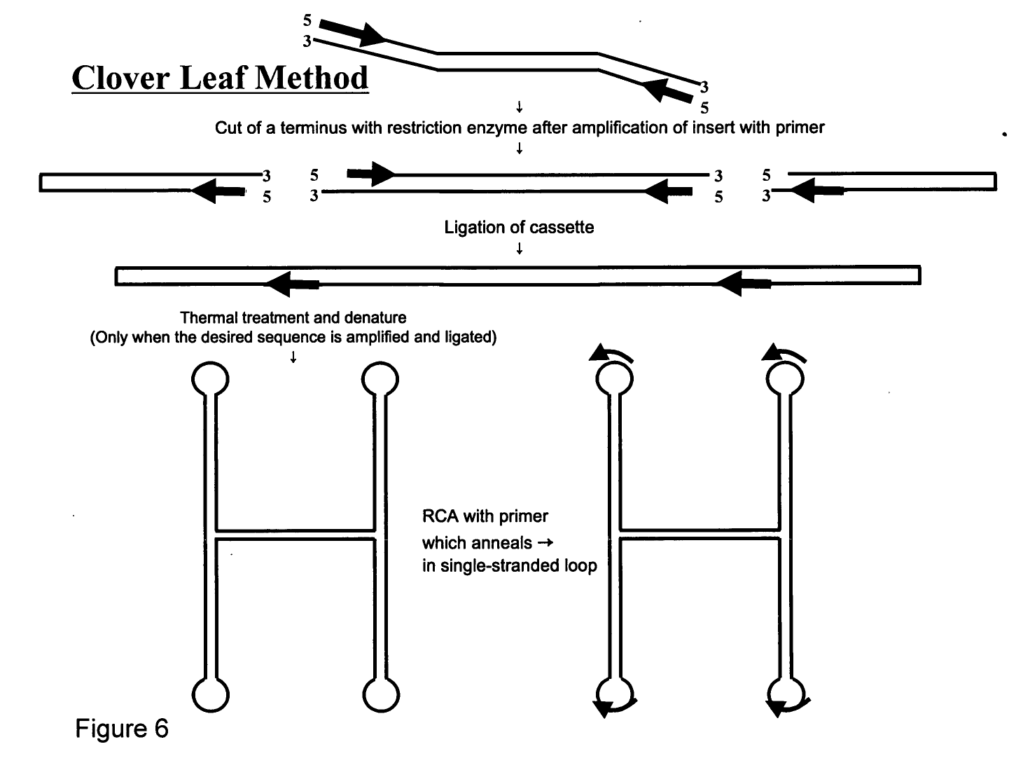

A mouse musculus achaete-scute complex homolog-like 3 (Drosophila) (Ascl3) gene, ID Number: NM_{—}020051 was tried to be amplified using a mouse genome DNA as a template by a Clover Leaf method shown in FIG. 6. A sequence of the insert is shown below (SEQ ID NO: 10). The underlined parts are sequences which anneal to a 3′ terminal side of a primer, and a restriction enzyme (BamHI) cleavage site is shaded. This insert is called template A.

[image]

Amplification was performed using primers shown below. Synthesis of the primers was performed using a DNA synthesizer Model 394 of ABI (Applied Biosystem Inc.).

<Primer Sequence Used in Amplification of Insert Sequence>

(SEQ ID NO:11)

YH-F1
TAACTATAACGGTCCTAAGGTAGCGAATGGACACCAGAAGCTACCC

(SEQ ID NO:12)

YH-R1:
TAACTATAACGGTCCTAAGGTAGCGATCAAATGACTCTCAGAGCCG

In the primer sequences, the underlined sequence is a sequence which anneals to the underlined sequence of the insert. An I-CeuI restriction enzyme recognition sequence (bold letter part) is added to the 5′ terminal region of each primer.

Using these primers, the insert was amplified by PCR. A PCR reaction solution and the number of cycles are as follows.

<PCR>

| Component | Final | |
|---|---|---|
| 10Xbuffer | 1X | |
| MgCl₂ | 2.5 | mM |
| dNTPs | 200 | μM |
| Primer F | 0.2 | μM |
| Primer R | 0.2 | μM |
| Template | 500 | ng |
| AmpliTaq | 1.25 | U |
| H₂O | up to 25 | μl |

<Reaction Condition>
94° C.; 2 minutes, (95° C.; 30 seconds, 65° C.; 1 minute, 72° C.; 1 minute), 35 cycles

After PCR amplification, unreacted primers were removed by Promega Wizard® SV Gel and PCR Clean-Up system to purify a desired amplification product.

Further, in order to demonstrate specificity of the present method, a template DNA (referred to as template B) having a nucleotide sequence, a part of which is different from a base sequence of a template A, was artificially prepared, amplified as in a template A, and an amplification product was purified. The sequence of a template B is shown below (SEQ ID NO: 13). The sequence part which is different from the template A is shown by a bold letter.

[image]

The terminal of each amplification product of template A and template B was subjected to restriction enzyme treatment with I-CeuI. The reaction reagent composition is as follows. Restriction enzyme treatment was performed at 37° C. for 3 hours.

<Reaction Reagent Composition>

| | | |
|---|---|---|
| I-CeuI Buffer | 5 | μl |
| I-CeuI | 1 | μl |
| Purified amplification product | 10 | μl |
| dH₂O | up to 50 | μl |

Using Promega Wizard® SV Gel and PCR Clean-Up system, an amplification product in which a terminal was cut with a restriction enzyme was purified.

An amplification product after purification was ligated to a loop cassette. The sequence of a loop cassette is shown below. This loop cassette is a 5′ terminal phosphorylated oligonucleotide. The underlined part is the loop part. Each loop cassette was designed so that a 3′ terminus had an overhang of four bases (shown by bold letter). Further, after the ligation of the loop cassette, the sequence to which an amplification primer annealed is boxed (including a sense strand and an antisense strand). In addition, the sequence corresponding to the aforementioned primer sequence is underlined and, further, the sequence part such that, after amplification including a desired region sequence, the primer binds to the loop cassette and, after thermal denaturation, the linking product can form a different structure (only when a desired nucleic acid is amplified, a region homologous to a sequence in a loop can be produced) is shaded. The reaction reagent composition is as follows, and the ligation reaction was performed at 16° C. for 90 minutes. Thereafter, using Promega Wizard® SV Gel and PCR Clean-Up system, the unligated short chain loop cassette was removed to purify the sequence ligated with the loop cassette.

<Loop Cassette Sequence>

[image]

[image]

Ligation Solution>

| | | |
|---|---|---|
| LOOP-F2 (10 μM) | 1 | μl |
| LOOP-R2 (10 μM) | 1 | μl |
| I-CeuI digested sample | 10 | μl |
| T4 DNA ligase buffer | 2 | μl |
| T4 DNALigase (NEB) | 2 | μl |
| dH₂O | up to 20 | μl |

Amplification was performed using the resulting dumbbell form-type product as a template. Template A and template B were thermally denatured at 95° C. for 5 minutes, thereafter, this was allowed to stand at room temperature for 5 minutes, and Rolling Circle Amplification was performed at room temperature (25° C.) for 4 hours using the following reagent composition. The primer sequence is as follows. The loop primer was designed so that it could anneal to a loop sequence, and amplification was performed.

<Loop Primer: RCA Primer Sequence>

PGAL1: TACCTCTATACTTTAACGTC (SEQ ID NO:16)

<Amplification Reagent Composition>

| | | |
|---|---|---|
| 10Xbuffer | 2.5 | μl |
| 100XBSA | 0.25 | μl |
| DMSO | 1.25 | μl |
| dNTPs (Final 140 μM) | 1.4 | μl |
| T4Gene32 (Amersham) | 0.5 | μl |
| Phi29Pol (NEB) | 2.0 | μl |
| Template (equivalent to about 10⁷ molecules) | 6.0 | μl |
| Each Primer (Final 0.4 μM) | 2 | μl |
| H₂O | up to 25 | μl |

In order to confirm whether a desired sequence was amplified or not, restriction enzyme treatment was performed at 37° C. for 2 hours using a restriction enzyme BamHI.

| | | |
|---|---|---|
| Amplification product | 2 | μl |
| BamHI (TAKARA Co., Ltd. 10 unit) | 2.5 | μl |
| BufferK | 1 | μl |
| dH₂O | up to 10 | μl |

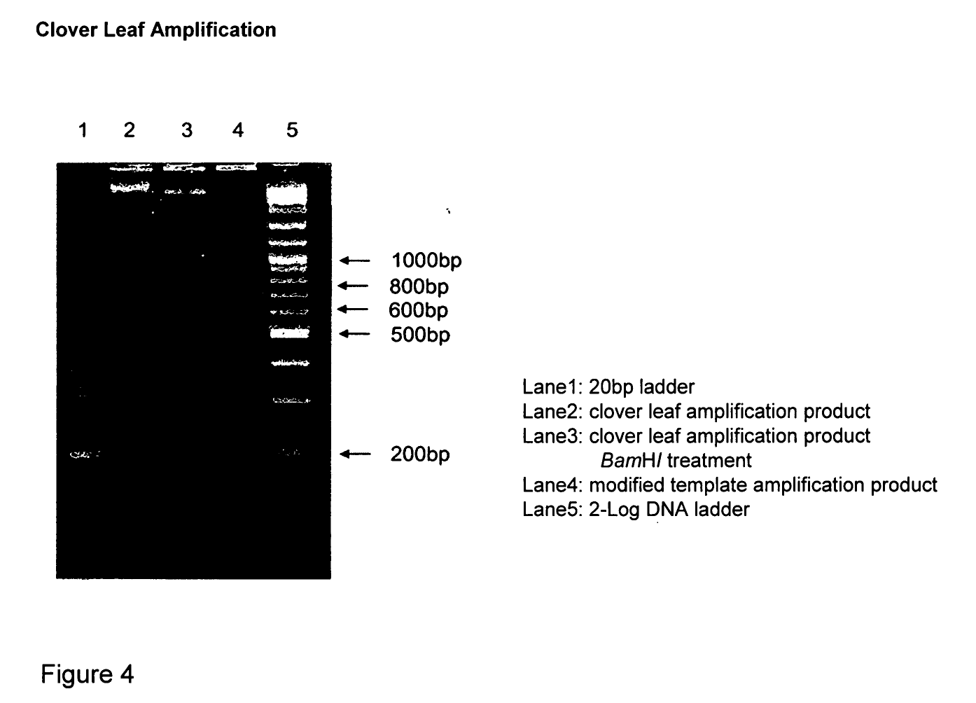

5 μl of the restriction enzyme-treated reaction solution was electrophoresed at 100V for 80 minutes on a 1.5% nusieve 3:1 agarose gel (manufactured by TAKARA SHUZO Co., Ltd.). A gel after electrophoresis was stained with ethidium bromide (EtBr) to confirm a nucleic acid. Results are shown in FIG. 4. Samples of respective lanes are shown in as follows.

Lane 1: 20 bp DNA Ladder size marker

Lane 2: amplification using template (A), and then untreatment with restriction enzyme

Lane 3: amplification using template (A), and then treatment with BamHI

Lane 4: amplification using template (B) of sequence change, after amplification, and untreatment with restriction enzyme

Lane 5: 2-Log DNA Ladder size marker

Lane 2 was untreated with the restriction enzyme, and the amplification product which had not been cut with the restriction enzyme was confirmed at about 10 Kbp. Lane 3 was cut with BamHI, and bands at about 600 bp and about 800 bp were confirmed. These results were consistent with the size predicted from a restriction enzyme map. From this, it was confirmed that amplification was performed using an insert sequence linked to the loop cassette as a template. However, amplification was not confirmed when template B in which a part of a sequence of template A was changed was amplified, and a loop cassette was bound thereto, and this was amplified as a template.

By the present method, it was found out that, specific amplification occurs only when a desired nucleic acid region (insert) is amplified, a loop cassette was bound thereto and, thereafter, a specific secondary structure can be formed.

### REFERENCES

Tsugunori Notomi et. al. (2000): Loop-mediated isothermal amplification of DNA. Nucleic Acids Research, Vol. 28, No. 12: e63
Kentaro Ngamine and Tesu Hase, Tsugunori Notomi: Accelerated reaction by loop-mediated isothermal amplification using loop primers. Molecular and Cellular Probes Vol. 16, No. 3, 223-229, 2002.

What is claimed is:

1. A method for amplifying a double-stranded nucleic acid, which comprises incubating the double-stranded nucleic acid in a solution containing at least one kind of a primer complementary to a part of one or more loop parts of a stem loop structure, under a condition where the double-stranded nucleic acid has the stem loop structure.

2. A method for amplifying a double-stranded nucleic acid, which comprises incubating the double-stranded nucleic acid in a solution containing at least one kind of a first primer and at least one kind of a second primer, under a condition where the double-stranded nucleic acid has a stem loop structure, wherein the first primer has a sequence complementary to a part of one or more loop parts of a stem loop structure and the second primer has a sequence complementary to an amplification product of the first primer.

3. A method for amplifying a double-stranded nucleic acid, which comprises steps of:
ligating a nucleic acid having at least one stem loop structure with the double-stranded nucleic acid; and
incubating the double-stranded nucleic acid in a solution containing at least one kind of a primer complementary to the part of one or more loop parts of a stem loop structure, under a condition where the double-stranded nucleic acid has the stem loop structure.

4. A method for amplifying a double-stranded nucleic acid, which comprises steps of:
ligating a nucleic acid having one or more stem loop structures with the double-stranded nucleic acid; and
incubating the double-stranded nucleic acid in a solution containing at least one kind of a first primer and at least one kind of a second primer, under a condition where the double-stranded nucleic acid has the stem loop structure, wherein the first primer has a sequence complementary to a part of one or more loop parts of a stem loop structure and the second primer has a sequence complementary to an amplification product of the first primer.

5. A method for amplifying a nucleic acid, which comprises steps of:
ligating an oligonucleotide forming a stem loop structure to one or more terminuses of a double-stranded nucleic acid, wherein the oligonucleotide contains any or both of a sequence complementary to a part of a first strand constituting a double-stranded nucleic acid, and a sequence complementary to a part of a second strand, and wherein the double-stranded nucleic acid can complementarily bind to the oligonucleotide to a part of the first strand, a part of the second strand or both of them, respectively, to form a new stem loop structure specific for a target double-stranded nucleic acid; and
incubating the nucleic acid in a solution containing at least one kind of a primer complementary to a loop part of the new stem loop structure.

6. A method for amplifying a nucleic acid, which comprises steps of:
ligating an oligonucleotide forming a stem loop structure to one or more terminuses of a target double-stranded nucleic acid, wherein the oligonucleotide contains either a sequence complementary to a part of a first strand constituting the double-stranded nucleic acid or a sequence complementary to a part of a second strand, or both of them and wherein the double-stranded nucleic acid can complementarily bind to the oligonucleotide to a part of the first strand, a part of the second strand, or both of them, respectively, to form a new stem loop structure specific for the double-stranded nucleic acid; and
incubating the nucleic acid in a solution containing at least one kind of a first primer and at least one kind of a second primer, wherein the first primer has a sequence complementary to a loop part of the new stem loop structure, and the second primer has a sequence complementary to an amplification product of the first primer.

7. A method for amplifying a nucleic acid, which comprises steps of:
ligating an oligonucleotide forming a stem loop structure to at least one or more terminuses of a target double-stranded nucleic acid, wherein the oligonucleotide contains either a sequence complementary to a part of a first strand constituting the double-stranded nucleic acid, or a sequence complementary to a part of a second strand, or both of them and wherein the double-stranded nucleic acid can complementarily bind to the oligonucleotide to a part of the first strand, a part of the second strand, or both of them, respectively, to form a new stem loop structure specific for the target double-stranded nucleic acid; and
incubating the nucleic acid in a solution containing at least one kind of a primer which is complementary to either a part of a first strand or a part of a second strand of the double-stranded nucleic acid constituting the loop part of the new stem loop structure, or both of them.

8. A method for amplifying a nucleic acid, which comprises steps of:
ligating a second nucleic acid to at least one or more terminuses of a target double-stranded nucleic acid containing one or more places of a single-stranded part forming a loop in a part of the double-stranded nucleic acid to form a hairpin structure or a loop structure; and
incubating the target double-stranded nucleic acid with the second nucleic acid linked thereto in a solution containing at least one kind of a primer complementary to a single-stranded part forming a loop in the double-stranded nucleic acid, or a part forming a loop at a terminus.

9. A method for amplifying a nucleic acid, which comprises steps of:
ligating a second nucleic acid to at least one or more terminuses of a double-stranded nucleic acid containing one or more places of a single-stranded part forming a loop in a part of a target double-stranded nucleic acid to form a hairpin structure or a loop structure; and
incubating the target double-stranded nucleic acid with the second nucleic acid ligated thereto in a solution containing one or more kinds of a first primer and one or more kinds of a second primer, wherein the first primer is complementary to a single-stranded part forming a loop in the target double-stranded nucleic acid or a part forming a terminal loop, and the second primer has a sequence complementary to an amplification product of the first primer.

10. The method for amplifying according to claim 9, wherein the double strand is derived from a double-stranded nucleic acid having a loop formed of complementary strands of two different nucleic acids which result from an alternative splicing.

11. A method for obtaining sequence information of two different nucleic acids from a nucleic acid which has been amplified by the method according to claim 10, wherein the two different nucleic acids result from an alternative splicing.

12. An oligonucleotide comprising a sequence complementary to a part of a target nucleic acid, wherein the oligonucleotide can form a secondary structure having one or more stem loop structures with the target nucleic acid, after the oligonucleotide is ligated to a target nucleic acid.

13. An oligonucleotide comprising a sequence complementary to a part of a first strand constituting a target double-stranded nucleic acid, and a sequence complementary to a part of a second strand, wherein a secondary structure having a new stem loop structure can be formed by binding complementarily the oligonucleotide and a part of the first strand or a part of the second strand or both of them, respectively, after the corresponding end of the double-stranded nucleic acid and two end of the oligonucleotide are ligated.


SEQUENCE LISTING (16 sequences)

SEQ ID NO: 1 - DNA, 525 bp, Mus musculus
atggacaccagaagctaccccagccctccagacaggctctcggtcttcgctgagtcggcccacttaccactgtccaggcccttctacctggaccccatggtcactgtccacctatgccccgagaccccggtaccggcctcttacacagatgagctgcctctgctgcccttctccagcgacaccctgatcatgaacaattacggggatccatacccttttcccttccccatgccttacaccaactacaggcgctgtgactacacctacgggccggccttcatccgcaagaggaacgagcgagagaggcaacgagtcaagtgtgtgaacgaaggctacgcccggctgcggcggcacctgccggaggactacctggagaaacggctcagcaaagtggagaccctcagagctgccatcaaatatatcagctacctgcagtctctcttgtacccggatgaatctgagaccaagaagaaccctcgaacagccagctgcggctccctggacccggctctgagagtcatttga

SEQ ID NO: 2 - DNA, 42 bp, Artificial Sequence
  Description of Artificial Sequence Synthetic Primer YH-F1
atgcgcggacccagattgctggatggacaccagaagctaccc

SEQ ID NO: 3 - DNA, 42 bp, Artificial Sequence
  Description of Artificial Sequence Synthetic Primer YH-R1
gctgcggcacccaacagaatggtcaaatgactctcagagccg

SEQ ID NO: 4 - DNA, 55 bp, Artificial Sequence
  Description of Artificial Sequence Synthetic Loop cassette sequence
gcatcgacggcatatgccatagcatttttatccacgatcacccgtcgatgcattg

SEQ ID NO: 5 - DNA, 54 bp, Artificial Sequence
  Description of Artificial Sequence Synthetic Loop cassette sequence
gagcctagcgcagtactgacgttaaagtatagaggtatcccgctaggctccaga

SEQ ID NO: 6 - DNA, 20 bp, Artificial Sequence
  Description of Artificial Sequence Synthetic Loop Primer pBADF
atgccatagcatttttatcc

SEQ ID NO: 7 - DNA, 20 bp, Artificial Sequence
  Description of Artificial Sequence Synthetic Loop Primer PGAL1
tacctctatactttaacgtc

SEQ ID NO: 8 - DNA, 20 bp, Artificial Sequence
  Description of Artificial Sequence Synthetic Stem Primer SF1
gatcacccgtcgatgcattg

SEQ ID NO: 9 - DNA, 20 bp, Artificial Sequence
  Description of Artificial Sequence Synthetic Stem Primer SR1
gtatcccgctaggctccaga

SEQ ID NO: 10 - DNA, 525 bp, Mus musculus; identical to SEQ ID NO: 1

SEQ ID NO: 11 - DNA, 46 bp, Artificial Sequence
  Description of Artificial Sequence Synthetic Primer YH-F1
taactataacggtcctaaggtagcgaatggacaccagaagctaccc

SEQ ID NO: 12 - DNA, 46 bp, Artificial Sequence
  Description of Artificial Sequence Synthetic Primer YH-R1
taactataacggtcctaaggtagcgatcaaatgactctcagagccg

SEQ ID NO: 13 - DNA, 525 bp, Artificial Sequence
  Description of Artificial Sequence Synthetic Template B
atggacaccagaagctacccctagctgatcttgcatcgtacggtcttcgctgagtcggcccacttaccactgtccaggcccttctacctggaccccatggtcactgtccacctatgccccgagaccccggtaccggcctcttacacagatgagctgcctctgctgcccttctccagcgacaccctgatcatgaacaattacggggatccatacccttttcccttccccatgccttacaccaactacaggcgctgtgactacacctacgggccggccttcatccgcaagaggaacgagcgagagaggcaacgagtcaagtgtgtgaacgaaggctacgcccggctgcggcggcacctgccggaggactacctggagaaacggctcagcaaagtggagaccctcagagctgccatcaaatatatcagctacctgcagtctctcttgtacccggatgaatctgagaccaagaagaaccctcgaacagctacgtgatcgtagctgatcgcggctctgagagtcatttga

SEQ ID NO: 14 - DNA, 152 bp, Artificial Sequence
  Description of Artificial Sequence Synthetic Loop-F2
tacctctatactttaacgtcctaaggtagcgaatggacaccagaagctaccccagccctccagacaggctctttttagagcctgtctggagggctggggtagcttctggtgtccattcgctaccttaggacgttaaagtatagaggtactaa

SEQ ID NO: 15 - DNA, 152 bp, Artificial Sequence
  Description of Artificial Sequence Synthetic Loop-R2
gacgttaaagtatagaggtactaaggtagcgatcaaatgactctcagagccgggtccagggagccgcagctgttttcagctgcggctccctggacccggctctgagagtcatttgatcgctaccttagtacctctatactttaacgtcctaa

SEQ ID NO: 16 - DNA, 20 bp, Artificial Sequence
  Description of Artificial Sequence Synthetic Primer PGAL1
tacctctatactttaacgtc